(19) United States
(12) Patent Application Publication
(10) Pub. No.: US 2025/0120916 A1
(43) Pub. Date: Apr. 17, 2025

(54) NANOTECHNOLOGY FOR CHEMOTHERA0PY DRUG CAPTURE

(71) Applicants: The Regents of the University of California, Oakland, CA (US); The Penn State Research Foundation, University Park, PA (US)

(72) Inventors: Amir Sheikhi, State College, PA (US); Alireza Khademhosseini, Los Angeles, CA (US)

(21) Appl. No.: 18/688,389

(22) PCT Filed: Oct. 4, 2022
(86) PCT No.: PCT/US2022/045628

§ 371 Date: Mar. 1, 2024

Related U.S. Application Data
(60) Provisional application No. 63/253,250, filed on Oct. 7, 2021.

(51) Int. Cl. A61K 9/14; A61F 2/02; A61K 31/717; A61P 39/00

(52) U.S. Cl. CPC A61K 9/14; A61F 2/02; A61K 31/717; A61P 39/00

(57) ABSTRACT

The instant disclosure teaches a highly efficient cellulose-based nanoadsorbent that can capture more than 6000 mg of doxorubicin (DOX), one of the most widely used chemotherapy drugs, per gram of the adsorbent at physiological conditions. Such drug capture capacity is more than 3200% higher than other nanoadsorbents, such as DNA-based platforms. The disclosure teaches how anionic hairy cellulose nanocrystals, also known as electrosterically stabilized nanocrystalline cellulose (ENCC), bind to positively charged drugs in human serum and capture DOX immediately without imposing any cytotoxicity and hemolytic effects. The disclosure further elucidates how ENCC provides a remarkable platform for biodetoxification at varying pH, ionic strength, ion type, and protein concentration. These discoveries pave the way for the next generation in vitro and in vivo drug capture additives and devices.

### CROSS-REFERENCE TO RELATED APPLICATIONS

This application claims the benefit under 35 U.S.C. Section 119(e) of co-pending and commonly-assigned U.S. Provisional Patent Application Ser. No. 63/253,250, filed on Oct. 7, 2021 and entitled “NANOTECHNOLOGY FOR CHEMOTHERAPY DRUG CAPTURE” which application is incorporated by reference herein.

### STATEMENT REGARDING FEDERAL FUNDING

This invention was made with government support under Grant Number EB024403, awarded by the National Institutes of Health. The government has certain rights in the invention.

### TECHNICAL FIELD

Embodiments of the disclosure concern at least the fields of medicine and pharmacology.

### BACKGROUND OF THE INVENTION

More than 39% of men and women are diagnosed with cancer at some stage in their lifetime(1) from whom more than 85% will suffer from one or more side effects,(2,3) including anemia, repeated infections, hair loss, jaundice, and fever. Despite the recent advances in drug-free cancer treatment methods, such as immunoengineering,(4,5) chemotherapy drugs remain the standard practice among clinicians.(6-8) As an example, an adult diagnosed with liver cancer may require 60, 75, and 800 mg/m² of doxorubicin (DOX),(9) cisplatin,(10) and 5-fluorouracil,(11) respectively, administered once a month via intravenous (IV) injection. Such treatment may result in anemia,(10,11) congestive heart failure,(9) cardiomyopathy,(9) seizures,(9,10) myelosuppression,(9-11) and mucositis.(9,11) in some cases, e.g., hepatocellular carcinoma (HCC), localized drug delivery approaches, such as catheter-based minimally invasive injection of chemotherapy drugs and drug eluting materials(12-18) may help increase the treatment efficacy; however, the unused drugs may still reach the circulation system and result in unpreventable damages to healthy tissues.(19,20)

To reduce the off-target effects of cancer drugs during and after localized chemotherapy, eliminating their systemic circulation is necessary. Recently, a few platforms have been proposed to remove unwanted drugs, mainly DOX, from blood, including an endovascular ion exchange device consisting of a porous nylon mesh cylinder filled with the ion exchange resin Dowex,(21) DNA-coated magnetic nanoparticles,(22) DNA-coated polyacrylate,(23) and block copolymers.(24,25) While these strategies have shown some success in removing DOX from blood, their removal efficacy remains exceedingly low, e.g., in the order of a few μg of DOX per mg of adsorbent within several minutes, often necessitating the use of large devices, e.g., ˜0.5 m long tubes,(22) to remove physiologically relevant concentrations of DOX

To overcome the challenges associated with current drug capture materials, specifically the low removal capacity, we considered nanoparticles with a large density of functional groups as promising candidates A large portion of therapeutic drugs, e.g, some chemotherapeutic agents, bactericides, fungicides, virucides, and opioids, contain positively charged moieties,(26) which may bind to oppositely charged substances via electrostatic interactions. However, due to the complex blood composition, charged nanoparticles typically lose their functionality in blood as a result of ionic strength and protein mediated aggregation via several mechanisms, such as electrical double layer screening and bridging.(27,28) Uncharged moieties, such as polyethylene glycol (PEG), have widely been used to protect nanoparticles in blood via steric repulsion and render them stealth;(29-31) however, they may result in compromised drug binding affinity as a result of reduced surface charge (32) To the best of our knowledge, there is currently no nanoparticle-based super-capacity drug capture system.

It is an object of the present invention to provide compositions, devices and methods useful for in vitro and in vivo drug capture.

### SUMMARY OF THE INVENTION

The invention disclosed herein provides a cellulose-based nanotechnology for removing excess chemotherapy drugs from the body to reduce the side-effects of cancer therapy. In particular, the instant disclosure describes a highly efficient cellulose-based nanoadsorbent that can capture more than 6000 mg of doxorubicin (DOX), one of the most widely used chemotherapy drugs, per gram of the adsorbent at physiological conditions (see, e.g., FIG. 4). Such drug capture capacity is more than 3200% higher than other nanoadsorbents, such as DNA-based platforms. The instant disclosure teaches how anionic hairy cellulose nanocrystals, also known as electrosterically stabilized nanocrystalline cellulose (ENCC), bind to positively charged drugs in human serum and capture DOX immediately without imposing any cytotoxicity and hemolytic effects. This disclosure includes elucidating how ENCC provides a remarkable platform for biodetoxification at varying pH, ionic strength, ion type, and protein concentration (see, e.g., FIG. 7). These discoveries pave the way for a next generation of in vitro and in vivo drug capture additives, devices and associated methods.

The invention disclosed herein has a number of embodiments that harness the above-noted discoveries. Embodiments of the invention include, for example, compositions of matter comprising electrosterically stabilized nanocrystalline cellulose, wherein the electrosterically stabilized nanocrystalline cellulose captures >6000 mg of doxorubicin per gram of electrosterically stabilized nanocrystalline cellulose when the electrosterically stabilized nanocrystalline cellulose is contacted with a fluid comprising doxorubicin. In certain embodiments of the invention, these compositions include further components such as a pharmaceutically acceptable carrier; a polypeptide having a negative charge at physiological pH (e.g. an albumin); blood; and/or a therapeutic agent such as doxorubicin. In some embodiments of the invention, the electrosterically stabilized nanocrystalline cellulose is coupled to a matrix (e.g. beads, nanoparticles or like matrices that are disposed within a depot/chamber of a medical device).

Embodiments of the invention also include medical devices designed to facilitate the use of the compositions of the invention. Illustrative medical devices can include, for example, a depot/chamber; an electrosterically stabilized nanocrystalline cellulose composition disposed within the depot; and a conduit having a first end adapted to capture a fluid and a second end coupled to the depot. Such medical devices can also include a second conduit having a first end coupled to the depot and a second end adapted to deliver a fluid out of the depot. Optionally, the device further comprises a housing which envelopes the depot, wherein the exterior surface of the housing comprises a biocompatible material adapted to be inserted in vivo.

Embodiments of the invention further include methods of removing doxorubicin from a fluid comprising doxorubicin, the methods comprising contacting an electrosterically stabilized nanocrystalline cellulose composition disclosed herein with the fluid such that the electrosterically stabilized nanocrystalline cellulose binds/captures the doxorubicin, thereby removing doxorubicin from the fluid. In certain embodiments of these methods, the fluid is blood. In some embodiments of the invention, the fluid is contacted in vivo.

There are a number of advantages of the novel cellulose nanotechnologies disclosed herein as compared to conventional nanoadsorbents. Nanoengineered cellulose provides a super-capacity drug nanoadsorbent, with dicarboxylated hairy cellulose nanocrystals having an ability to capture >6000 mg of DOX per gram, a drug capture capacity is 3 orders of magnitude higher than DNA-based capture technologies. For these reasons, hairy nanocelluloses provide a remarkable platform for biodetoxification and enable a next generation of drug capture devices.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. ENCC synthesis and characterization. (a) ENCC synthesis involving two-step oxidation using periodate to initially open the glucose ring from C2-C3 bond, converting them to dialdehyde groups, followed by chlorite-mediated oxidation to carboxylate groups. AFM images of (b) CNC and (c) ENCC. Titration of ENCC (20 mg) based on (d) conductivity and (e) pH versus added base (NaOH, 10 mM) volume. The inset in panel (e) shows the first and second derivatives of pH versus NaOH volume.

FIG. 2. Drug capture in aqueous media. (a) DOX removal R=(C₀−C_{g}/C₀ by ENCC or CNC in DPBS versus initial DOX concentration C₀, obtained from measuring the equilibrium DOX concentration Cₑ using a microvolume UV-vis spectrophotometer. (b) DOX capture capacity of ENCC (black symbols) or CNC (blue symbols) defined as qₑ=(C₀−Cₑ) Vlm_{ENCC} versus equilibrium DOX concentration Cₑ and the corresponding Langmuir fit (curve). Sample volume V˜500 μL. The DOX removal capacity of ENCC in DPBS is >5200 μg mg⁻¹, and CNC is unable to effectively capture DOX due to the lack of high-density anionic groups. The background error is corrected. (c) Optical images of DOX solutions with concentrations ranging from ˜300 μg mL⁻¹ to ˜1400 μg mL⁻¹ before and after the addition of ENCC with a final concentration ˜195 μg mL⁻¹. This wide range of concentrations corresponds to a broad ratio of DOX to the carboxylate content of ENCC (˜0.46-2.07). (d) Time-dependent DOX capture using ENCC, showing an extremely fast drug removal taking place almost immediately after ENCC-DOX contact. Here, DOX concentration (DOX) ˜311, 622, and 1244 μg m⁻¹, and ENCC concentration (ENCC) ˜195 μg mL⁻¹, and the ratio of DOX to the COO— of ENCC ˜0.47, 0.92, 1.82 mol mol⁻¹. (e) Colloidal properties of DOX significantly change upon adsorption onto ENCC. Hydrodynamic size of DOX aggregates ˜66 nm increases to ˜350 nm, an indication of ENCC-mediated aggregation. The ζ-potential of colloidal DOX changes from ˜+24 mV to ˜−32 mV as a result of adsorption to the negatively-charged ENCC. Note that (DOX)(COO⁻)˜0.1, therefore ENCC (concentration 100 μg mL⁻¹) is not saturated, forming stable colloidal aggregates.

FIG. 3. Effects of pH and ionic strength on ENCC-mediated DOX capture. (a) DOX removal percentage versus initial DOX concentration, and (b) DOX capture capacity of ENCC versus equilibrium DOX concentration at pH˜4. Decreasing pH to 4 resulted in the protonation of carboxylic acid groups, which decreased the available DOX binding sites on ENCC, decreasing the removal percentage and capture capacity. The capture capacity at pH ˜4 is less than half of that at pH˜7.4 (FIG. 2b). The effect of monovalent ion, Na⁺, on the (c) removal percentage and (d) capture capacity of ENCC, showing no significant impact because the equilibrium of carboxylate groups is not affected by monovalent ions. Effect of divalent ion, Ca²⁺, on the (e) removal percentage and (f) capture capacity of ENCC. The presence of 0.9 and 90 mM of Ca²⁺ decreased the DOX capture capacity of ENCC by a factor of 35% and 600%, respectively. This proves that the main drug capture mechanism is DOX-ENCC electrostatic attraction, disturbed by the divalent ion-mediated bridging of ENCC. In panels b, d, and f, the curve shows the Langmuir fit.

FIG. 4. ENCC-mediated DOX capture in physiological media. Effect of BSA on the DOX (a) removal percentage and (b) capture capacity of ENCC, showing that the serum albumin not only does not impair the drug capture capability of ENCC, but also enhances the removal capacity as a result of DOX-protein binding. The DOX (c) removal percentage and (d) capture capacity of ENCC in human serum. The DOX capture capacity of ENCC in human serum is remarkably high, possibly as a synergistic effect of drug-protein complex formation. (e) The drug capture time scale in human serum is extremely short, allowing for almost immediate removal of the chemotherapy drug. (f) Increasing the ENCC concentration increases the DOX removal capacity, allowing for almost complete elimination of the drug.

FIG. 5. ENCC cytotoxicity.(a) Effect of ENCC concentration on the HUVECs, shown with live (green)/dead (red) and F-actin (green)/DAPI (blue) staining after 24 h and 72 h post nanoparticle exposure, respectively. Almost all the cells are viable and have undergone spreading and elongation with no significant damage to their nuclei. The scale bars are 200 μm. (b) Metabolic activity of HUVECs after 72 h exposure to various concentrations of ENCC normalized with the metabolic activity in the absence of ENCC, obtained using the PrestoBlue™ assay. The unchanged metabolic activity of HUVECs show that ENCC is not toxic against endothelial cells.

FIG. 6. Assessment of the hemolytic properties of ENCC. (a) Optical images of blood exposed to varying concentrations of ENCC compared with the negative control (NC. PEG) and positive control (PC, Triton X-100). The disruption of RBC is seen as stable red color in the supernatant post-centrifugation. Up to 7 mg mL of ENCC does not have any significant effect on the color of supernatant attesting to the integrity of RBCs. (b) Quantification of hemolysis percentage via measuring the absorbance of supernatant. While the positive control vigorously disrupts the RBCs resulting in >95% hemolysis, up to 7 mg mL⁻¹ of ENCC yields less than 4% hemolysis, which is below the accepted threshold (5%).

FIG. 7. In vitro biodetoxification effect of ENCC. (a) Live/Dead staining of HUVECs and NIH/3T3 fibroblasts after 8 h culturing in cell culture media (control), ENCC (1 mg mL⁻¹)-treated DOX (400 μg mL⁻¹)-containing media, and untreated DOX (400 μg mL⁻¹)-containing media. The scale bars are 200 μm. Normalized viability of (b) HUVECs and (c) NIH/3T3 fibroblasts calculated from analyzing the fluorescence images in panel (a). Metabolic activity of (d) HUVECs and (e) NIH/3T3 cultured in similar conditions as (a), measured using the PrestoBlue™ cell viability reagent.

FIG. 8. Medical device comprising electrosterically stabilized nanocrystalline cellulose.. This figure provides a schematic of a device including a depot in which the electrosterically stabilized nanocrystalline cellulose is disposed, wherein the depot is coupled to a first conduit adapted to facilitate fluid flow from an external environment to the electrosterically stabilized nanocrystalline cellulose and also a second conduit adapted to facilitate fluid flow away from the electrosterically stabilized nanocrystalline cellulose within the depot.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

Cancer is one of the leading causes of death worldwide, affecting millions of people every year. While chemotherapy remains one of the most common cancer treatments in the world, the severe side effects of chemotherapy drugs impose serious concerns to cancer patients. In many cases, the chemotherapy can be localized to maximize the drug effects; however, the drug systemic circulation induces undesirable side etTects.

Among nanoparticles, nanoscale celluloses, known as nanocellulose, are an interesting candidate to develop drug capture systems due to their benign chemical structure,(33,34) non-cytotoxic nature,(35-40) and non-biodegradability(41) in the human body. Conventional nanocelluloses are commonly classified as cellulose nanocrystals (CNC) and cellulose nanofibrils (CNF), neither of which may accommodate more than 2 mmol of anionic functional groups, such as carboxylates, per gram due to the inaccessibility of their inner crystalline cellulose chains.(42-48) The emergence of hairy cellulose nanocrystals,(49-51) CNCs decorated with highly functionalized amorphous cellulose chains (hairs) at both ends that are synthesized via the controlled oxidation of cellulose fibers, has opened new opportunities for engineering nanoadsorbents(52) with significantly higher removal capacities than other bio-based materials.

Here, we describe nanoengineering cellulose to yield anionic hairy cellulose nanocrystals, also known as electrosterically stabilized nanocrystalline cellulose (ENCC), for removing one of the most common chemotherapy drugs, DOX as a model cationic drug with significant side effects,(53-56) in physiological conditions. We investigate the mechanism of drug removal via altering ionic strength, pH, and protein content, and quantify the drug removal efficacy. The ENCC is compared with CNC to shed light on the fundamental advantages of hairy nanocelluloses over conventional ones. In addition, in vitro cytotoxicity and hemocompatibility of ENCC is investigated.

The invention disclosed herein has a number of embodiments that harness and build upon the above-noted discoveries. Embodiments of the invention include, for example, pharmaceutical compositions of matter comprising electrosterically stabilized nanocrystalline cellulose (see, e.g., Yang et al., Langmuir 28(20):7834-7842, 2012; and Sheikhi et al., J. Vis. Exp. 113 (2016.) e54133. doi:10.3791/54133), wherein the electrosterically stabilized nanocrystalline cellulose binds/captures at least 1000 mg, 2000 mg, 3000 mg, 4000 mg, 5000 mg, or 6000 mg of doxorubicin per gram of electrosterically stabilized nanocrystalline cellulose when the electrosterically stabilized nanocrystalline cellulose is contacted with a fluid (e./g. blood) comprising doxorubicin. In certain embodiments of the invention, such compositions include further components such as a pharmaceutically acceptable carrier; a polypeptide having a negative charge at physiological pH (e.g. an albumin); blood, and/or therapeutic agent such as doxorubicin. For compositions suitable for use in humans, the term “excipient” is meant to include, but is not limited to, those ingredients described in Remington: The Science and Practice of Pharmacy, Lippincott Williams & Wilkins, 21st ed. (2006) the contents of which are incorporated by reference herein. Common illustrative excipients include antimicrobial agents and buffering agents. In some embodiments of the invention, the electrosterically stabilized nanocrystalline cellulose is coupled to a matrix (e.g beads, nanoparticles, resins or the like). Optionally, the electrosterically stabilized nanocrystalline cellulose is disposed with a depot of a medical device.

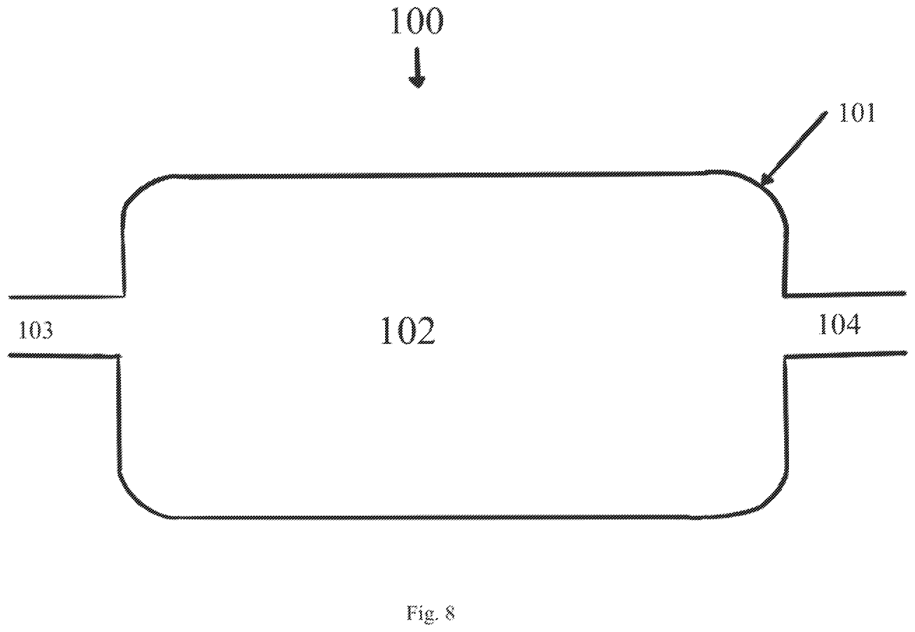

Embodiments of the invention also include medical devices designed to facilitate the use of the compositions of the invention (e.g., to remove excess doxorubicin from the blood of a cancer patient being treated with doxorubicin). As shown in FIG. 8, illustrative medical devices (100) can include, for example, a depot/chamber (101); an electrosterically stabilized nanocrystalline cellulose composition (102) disclosed herein disposed within the depot; and a conduit (103) having a first end adapted to capture a fluid and a second end coupled to the depot so as to direct the fluid to the electrosterically stabilized nanocrystalline cellulose composition Such medical devices can also include a second conduit (104) having a first end coupled to the depot and a second end adapted to deliver a fluid out of the depot. In certain embodiments of the invention, the electrosterically stabilized nanocrystalline cellulose within the medical device comprises blood and/or doxorubicin. Optionally, the device further comprises a housing which envelopes the depot, wherein the exterior surface of the housing comprises a biocompatible material adapted to be inserted in vivo. In certain embodiments of the invention, the device is used in a patient is selected to be one diagnosed as having a cancer of the breast, bladder, kidney, ovary, thyroid, stomach, lung, bone, nerve tissue, joint, or soft tissue or in a patient diagnosed as having a lymphoma or a leukemia.

In one medical device embodiment of the present invention, a catheter or probe for extracting doxorubicin from blood circulating in a patient may comprise an elongate body having a proximal end and a distal end Usually, at least the distal end is configured to be introduced into a blood vessel lumen of the patient. The elongate body can have a chamber, usually within the distal end of the elongate body where the chamber is configured to allow blood to flow through when the catheter is present in the blood vessel lumen. The chamber can hold the electrosterically stabilized nanocrystalline cellulose of the present invention as described above. Exemplary catheters useful in the present invention are described in WO2014/100201, the full disclosure of which is incorporated herein by reference. The catheters may be positioned in different blood vessels including the hepatic vein, iliac vein, inferior vena cava, renal vein, and superior vena cava. Additional exemplary locations include, intracranial in the dural venous sinuses (e.g., sigmoid sinus, transverse sinus, torcula, straight sinus, superior sagittal sinus) to remove doxorubicin during cerebral embolization or chemoinfusion; internal jugular vein with the device inserted, for example, either transfemorally or directly in the ipsilateral internal jugular vein, for head and neck tumors and during cerebral embolization or chemoinfusions; and the brachiocephalic vein between the superior vena cava and the internal jugular vein.

Embodiments of the invention further include methods of removing doxorubicin from a fluid comprising doxorubicin, the methods comprising contacting an electrosterically stabilized nanocrystalline cellulose composition disclosed herein with the fluid such that the electrosterically stabilized nanocrystalline cellulose binds/captures the doxorubicin, thereby removing doxorubicin from the fluid. In certain embodiments of these methods, the fluid is blood. In some embodiments of the invention, the fluid is contacted in viro. In illustrative embodiments of the invention, blood can be exposed to the electrosterically stabilized nanocrystalline cellulose material, e.g, this material disposed in/on a matrix or resin, such as beads of the type used in ion exchange, or a membrane, or a filter substrate or the like, for time sufficient to extract at least a portion of the doxorubicin present in the blood, often for a time sufficient to remove substantially all of the doxorubicin present in the blood.

Further aspects and embodiments of the invention are discussed in the following sections.

### Materials and Methods

### Materials

Doxorubicin hydrochloride (DOX) powder was purchased from Oakwood Chemical (USA), cellulose nanocrystal (CNC, freeze-dried solid, including 0.85 wt % sulfur on dry CNC sodium form) was provided by the University of Maine Process Development Center (USA), human serum (sterile filtered male AB plasma), hydrochloric acid (HCl, 37%), bovine serum albumin (BSA, lyophilized powder, 66 kDa), sodium hydroxide (NaOH, ACS reagent, ≥97%), sodium (meta)periodate (NaIO₄, ≥99.99%), sodium chlorite (NaClO₂, 80%), sodium chloride (NaCl, ACS reagent, ≥99%), ethylene glycol (C2H602, 99%), polyethylene glycol (PEG, Mw=400 Da), calcium chloride (CaCl₂, 99.99%), poly-L-lysine (PLL) solution (0.01%, sterile-filtered), Triton X-100, Drabkin's solution, Alfa Aesar™ paraformaldehyde (4% in PBS), and pure human hemoglobin (lyophilized powder) were supplied by Sigma-Aldrich (USA). Milli-Q water (18.2 MΩ cm at 25° C.) was provided by the Millipore Corporation (USA). Disposable Malvern PANalytical Inc. (DTS1070) folded capillary cells, hydrogen peroxide (H2O2, 30%), and anhydrous ethanol (C2H50H, 95.27%) were purchased from Fisher Scientific (Canada and USA). Bleached softwood (black spruce) kraft pulp sheets (Q-90) were provided by FPInnovations (Canada). The dialysis membrane (12-14 kDa molecular weight cutoff) was purchased from Spectrum Lab Inc (USA). NIH/3T3 fibroblast cells and human umbilical vein cells (HUVECs) were purchased from the American Type Culture Collection (ATCC, USA). Heat inactivated fetal bovine serum (FBS), Dulbecco's phosphate-buffered saline (DPBS, 1×), Dulbecco's modified Eagle medium (DMEM, including 4.5 g of glucose per liter), trypsin-ethylenediaminetetraacetic acid (trypsin-EDTA, 0.5%, 1×), penicillin/streptomycin (P/S, 100×), and trypan blue solution (04%) were procured from Gibco (USA). PrestoBlue™ cell viability reagent, Live/Dead™ viability/cytotoxicity kit, and Alexa Fluor™ 488 Phalloidin were produced by Invitrogen and provided by ThermoFisher Scientific (USA), and 4′,6-diamidino-2-phenylindole (DAPI) was purchased from ThermoFisher Scientific. Endothelial cell growth medium-2 (EGM-2) Bulletkit was purchased from Lonza. Tissue culture flasks (75 cm2) were purchased from Corning (USA), and tissue culture-treated polystyrene 96-well plates were obtained from Falcon (USA).

ENCC synthesis: ENCC is an anionic hairy cellulose nanocrystal that bears a large amount of dicarboxylates groups on its hairs. We prepared ENCC via the chlorite-mediated oxidation of dialdehyde modified cellulose (DAMC) fibers based on our previous protocols.(50,57) These fibers were prepared by soaking 4 g (25 mmol glucose units) of small (˜2 cm2) softwood pulp pieces in water overnight, which were disintegrated mechanically using a blender. The pulp slurry was oxidized via a reaction with an aqueous periodate (5.33 g) solution including ˜266 g water (absorbed water by pulp+added water) in the dark to prevent periodate deactivation. The reaction was stopped after 4 days using 3 mL of ethylene glycol, and the intact DAMC fibers were rinsed with water five times and vacuum filtrated. To synthesize ENCC, 1 g of DAMC fibers was reacted with 1.41 g of sodium chlorite in an aqueous solution containing 1.41 g of hydrogen peroxide and 2.93 g of sodium chloride (total water volume ˜266 mL) The pH was maintained at ˜5 via intermittent NaOH (0.5 M) addition for 24 h. After the reaction completion, 0.16 g of ethanol per 1 g of mixture was added, and ENCC was separated via centrifugation (3000 g for 10 min). ENCC was then dialyzed against DI water for at least one day to remove the impurities and salts.

Conductometric titration. To measure the carboxylate content of nanocelluloses, we used an established protocol.(58) A dispersion of never-dried ENCC containing 20 mg of solid in 140 mL of Milli-Q water was prepared to which 2 mL of a 20 mM NaCl solution was added, followed by adjusting the pH to 3 using a solution of 0.1 M HCl. The ENCC dispersion was titrated using the Methrom 907 Titrando instrument to add a 10 mM NaOH solution at a rate of 0.1 mL/min until reaching pH ˜11. The carboxylate group density of ENCC was calculated based on the NaOH volume required to neutralize the weak acid, i.e., the middle region in the titration curve.

Atomic force microscopy (AFM) imaging: To image CNC and ENCC, we adopted the sample preparation and imaging parameters from literature.(59) In brief, a PLL solution (0.1% w/v) was placed on a freshly cleaved mica disc for 5 min, rinsed with Milli-Q water, and the disc was mounted on a magnetic stainless steel disc. This is a necessary treatment to ensure the deposition of negatively charged nanocelluloses on a positively charged polymer. To deposit the nanocelluloses, a droplet of dispersion (concentration=0.1 mg mL−1) was incubated on the PLL-treated mica for 5 min, followed by the removal of excess liquid via gentle Milli-Q water rinse and air-drying overnight. The samples were imaged under the tapping mode using an AFM (Bruker Dimension Icon) equipped with a silicon nitride probe (Scanasyst-Air). For image processing, the NanoScope Analysis software (version 2.0) was used, and the nanocellulose dimensions were measured using Gwyddion (version 2.56).(60)

Hydrodynamic size and ζ-potential measurements: A Zetasizer Nano ZS from Malvern Panalytical Ltd was used to conduct dynamic light scattering (DLS) and electrokinetic measurements in folded capillary cells (DTS1070). Samples were prepared with an ENCC concentration of 0.1 mg mL−1 in 1 mg mL−1 of NaCl solution A final DOX concentration of 0.03 mg mL−1 was used to have an ˜1:10 molar ratio of DOX to the carboxylate of ENCC For ζ-potential measurements on DOX, the concentration was lowered to 0.003 mg mL−1. At least three measurements were taken for each DLS experiment, with 11 runs per measurement. For the ζ-potential tests, the number of measurements was at least three, with up to 100 runs per measurement.

DOX capture: DOX capturing experiments were carried out in varying media. Each sample had a total volume of 500 μL, except the removal tests in human serum for which 250 μL was used. The concentration of ENCC was fixed at 195 μg mL−1 for all samples. The media included DPBS, Milli-Q water with adjusted pH 4 using HCl, Milli-Q water with adjusted monovalent ion concentration (171 mM) using an NaCl solution, Milli-Q water with adjusted divalent ion concentration (0.9 or 90 mM) using a CaCl₂ solution, DPBS including BSA (35 mg mL−1), and human serum. Samples were prepared by adding the nanocellulose dispersion to a DOX solution at DOX: COO— of nanocellulose ratios between˜0.46-2.07 mol mol-1, covering ratios well below and above the stoichiometric (1:1 mol mol-1) value. The concentration of DOX in the supernatant of each sample was measured after centrifuging the samples for 2 min at 2000 rpm and 20° C. using 2 μL aliquots tested by a the NanoDrop One microvolume spectrophotometer (ThermoFisher Scientific, USA) at 480 nm. Standard curves were obtained for the freshly prepared DOX stock solutions in the same media as the removal tests, which were used to quantify the free drug in the supernatant after adding the nanocelluloses. For the time dependent measurements, the samples were centrifuged at pre-defined time points, aliquots were withdrawn and analyzed under the same conditions as equilibrium experiments. For control experiments, nanocellulose dispersions were replaced with an equivalent volume of MilliQ water.

In vitro cytotoxicity tests: The cytotoxicity of ENCC against HUVECs was assessed by incubating the nanoparticles with two-dimensionally (21)) cultured cells. HUVECs were cultured in EGM-2 Bulletkit cell culture medium at 37° C. and 5% CO2, followed by trypsinization using 0.5% trypsin-EDTA and seeding into 96-well plates at a density of 2.5×103 cells per well. After cell adhesion ˜2 h post seeding, the culture media were replaced with 50 μL of complete media containing varying ENCC concentrations. ENCC-containing media and ENCC-free media (control) were refreshed every other day. Cell viability was assessed after 1 day of exposure to ENCC via incubating the cells in a solution of DPBS including 0.05% v/v calcein AM (green staining of live cells) and 0.2% v/v ethidium homodimer (red staining of dead cells) (Live/Dead viability kit, Invitrogen) for 10 min. Following the incubation, the cells were washed twice with DPBS and maintained in 50 μl of DPBS to be examined under a Zeiss fluorescence microscope (Axio Observer 5, Zeiss, Germany). To examine the morphology, cells were fixed at day 3 with 4% v/v paraformaldehyde for 15 min and washed three times with DPBS. A solution of 0.3% v/v Triton X-100 in DPBS was used to permeabilize the fixed cells while a solution of BSA (1% w/v) in DPBS was used for 30 min at room temperature to block non-specific binding. The cells were then maintained at 4° C. until the final staining procedure was completed. The cytoskeleton was stained with a 1:40 dilution of Alexa Fluor 488 in a BSA solution (1% w/v) and incubated for 40 min at room temperature. Subsequently, the nuclei were counterstained with a 1:1000 dilution of DAPI in DPBS and incubated for 15 min at room temperature in dark. Images were acquired using a Zeiss fluorescence microscope at the excitation/emission wavelengths of ˜494/515 nm (calcein), ˜528/617 nm (ethidium homodimer-1), ˜495/518 nm (Alexa Fluor™ 488 Phalloidin), and 358/461 nm (DAPI). The metabolic activity of HUVECs was measured after 3 days of exposure to ENCC. Cells were incubated with 10% PrestoBlue™ cell viability reagent for 90 min in the culture media at 37° C. and 5% CO2. Subsequently, the supernatant was collected from each sample and transferred to 96-wells plates. The fluorescence (excitation/emission wavelengths of ˜530/590 nm) of supernatant was determined using the BioTek Synergy 2 microplate reader (USA). Measurements were performed in triplicate and the background signal (PrestoBlue™-containing cell-free media) was subtracted from the fluorescence values.

Hemolysis assessment: The hemolytic activity of ENCC was measured via exposing human whole blood, withdrawn from 3 healthy volunteer donors by another lab, to varying concentrations of ENCC and measuring the concentration of released hemoglobin in plasma according to the American Society for Testing and Materials (ASTM) E2524-08 (2013) standard analysis of hemolytic activity of nanoparticles.(61) A colorimetric technique was used to measure the oxidation of methemoglobin by cyanide (Drabkin's reagent) at an absorbance of 540 nm A calibration curve was obtained using 0.025-1 mg/t of pure human hemoglobin to quantify the concentration of released hemoglobin in heparinized human whole blood exposed to ENCC The blood samples were diluted in DPBS to have 10±2 mg of hemoglobin per mL. ENCC was added to a microcentrifuge tube, followed by the addition of DPBS (800 μL) and the diluted blood (100 μL). After gently mixing the samples, they were incubated for 3 h±15 min at 37° C. and centrifuged for 15 min at 14000 rpm and ambient temperature. A portion of supernatant (˜100 μL) was pipetted into a 96-well plate and mixed with 100 μL of the Drabkin's reagent. The reaction was continued for 15 min at room temperature in dark. A microplate reader (540 nm, BioTek UV/vis Synnergy 2, USA) was then used to measure the relative absorbance of each well against the reagent blank to correct the background (blood-free samples) absorbance. The hemolysis percentage was measured based on the hemoglobin concentration in the samples divided by the total blood hemoglobin (˜10 mg mL−1). Positive and negative controls were Triton X-100 (1% v/v) and PEG (4.4% v/v), respectively.

Biodetoxification tests: To determine the biodetoxification effect of ENCC, 5×103 NIH/3T3 cells or HUVECs were seeded in 96-well plates with 200 μL of DMEM supplemented with FBS (10% v/v) and 1% v/v of Pen-Strep or EGM bullet kit HUVEC media, respectively. The cells were allowed to attach to the bottom of wells for 1 h, the media were aspirated, and the cells were rinsed with 200 μL of DPBS, followed by the addition of 200 μL of corresponding media including DOX (final concentration=400 μg mL−1), which was previously detoxified by adding ENCC (final concentration=1 mg mL−1), centrifuged at 1000 rpm for 3 min, and separated from the precipitate. The control experiments were conducted in corresponding DOX-free cell culture media or DOX-containing media without ENCC treatment. Each group consisted of 5 wells. After 8 h, the media was aspirated, the cells were rinsed twice with 200 μL of DPBS, followed by the addition of 100 μL of PrestoBlue™ reagent to each well. The cells were incubated for 1 h, and then the PrestoBlue™ absorbance was recorded using the microplate reader. Immediately afterwards, the cells were again rinsed twice with 200 μL, of DPBS, followed by the addition of 150 μL, of the Live/Dead reagents. The cells were incubated for 90 min and imaged using the fluorescence microscope. The fluorescence images were analyzed using the Imagej Software.(62)

Statistical analysis: The data of at least three measurements are presented as mean values±standard deviation. We performed the two-way ANOVA (analysis of variance) and identified the statistically significant data when p≤0.05. Statistically significant values are presented as *p≤0.05, **p≤0.01, ***p≤0.001 and **** p≤0.0001.

### RESULTS AND DISCUSSION

### Engineering Hairy Celluose Nanocrystals

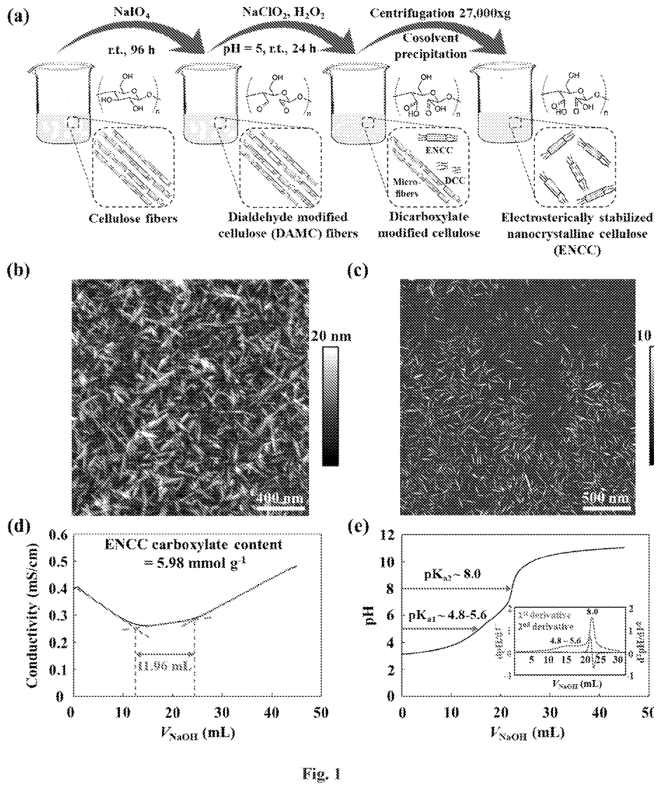

FIG. 1a shows the schematic of ENCC production from cellulose fibers via a two-step periodate-chlorite oxidation. The first reaction results in the production of intact DAMC fibers, which are then chemically disintegrated via chlorite-mediated oxidation of aldehyde groups to carboxylate, yielding a mixture of ENCC, dicarboxylated cellulose (DCC), and unfibrillated cellulose (micro-fibers). ENCC is separated from the system via ethanol-mediated precipitation. AFM images of conventional CNC and ENCC are shown in FIGS. 1b and 1c, respectively, attesting to a similar crystalline body. The length and diameter (height) of CNC are 153.9±43.7 nm and 6.2.2.1 nm, respectively, which are comparable with ENCC length, 104.5±29.7 nm, and diameter, 3.5±1.3 nm. The conductometric titration of ENCC, presented in FIG. Id, attests to about 5.98 mmol of carboxylate groups per gram of ENCC. This is about 600% higher than the conventional 2,2,6,6-tetramethylpiperidine 1-oxyl, 2,2,6,6-tetramethyl-1-piperidinyloxy (TEMPO)-mediated oxidized CNC.(63) We did not register any weak acid in the titration of CNC. During the conductometric titration, we recorded the solution pH as NaOH was added. The dispersion pH versus NaOH volume is presented in FIG. 1e with insets showing the first and second derivates of pH with respect to NaOH volume. According to this figure, two pKa values are registered for ENCC, associated with the adjacent carboxylate groups on the C2 and C3 of glucose rings, i.e, dicarboxylate groups ENCC is an anionic hairy cellulose nanocrystal, which benefits from electrostatic and steric colloidal stability due to the highly negatively charged carboxylate groups and excluded volume of hairs, respectively, thus called electrosterically stabilized.

### Chemotherapy Drug Capture in DPBS

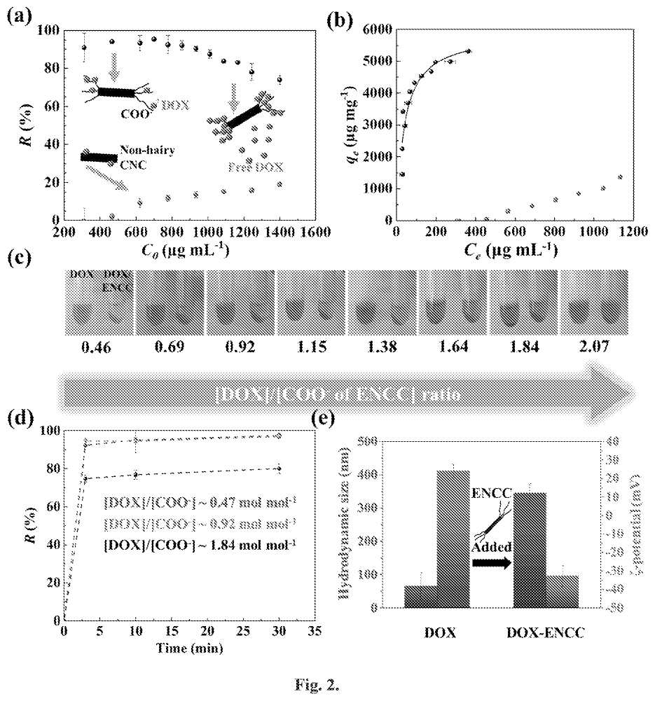

To study the interactions between the nanoadsorbent ENCC and chemotherapy drug DOX. ENCC was added to DOX solutions in DPBS with varying initial DOX concentrations (C0). The concentration of DOX was tuned in a way that the molar ratio of DOX to the negatively charged groups (COO— ˜5.98 mmol g−1) of ENCC varied from ˜0.46 to ˜2.07. FIG. 2a shows the removal percentage of DOX, R (%)=100×(C0−Ce)/C0, versus the initial DOX concentration (C0) when the nanoadsorbent concentration is ˜195 μg mL−1 (COO— concentration 1.166 mM). The equilibrium DOX concentration (Ce) was measured via sampling the supernatant after centrifuging ENCC-DOX samples. When the drug concentration was ≤800 μg mL−1 (˜1.4 mM), more than 90% of the drug was removed by the ENCC. Increasing the drug concentration beyond 800 μg mL−1 saturated the nanoadsorbent, decreasing the removal percentage to ˜70% at a DOX concentration of 1400 μg mL−1. The inset of FIG. 2a schematically shows the non-saturated and saturated states of ENCC. When the carboxylate groups of ENCC were not fully saturated by the positively-charged drug, most of the drug molecules were captured. Increasing the drug concentration eventually saturated the charged functional groups of ENCC, decreasing the drug removal percentage. As a control, the drug capture capability of conventional, non-hairy CNC was evaluated. CNC is commonly prepared by the strong acid (sulfuric acid)-mediated hydrolysis of cellulose fibers (64) As can be seen in FIG. 2a, the removal percentage of CNC is below 20%, and it is unable to effectively capture DOX as a result of the low density of charged groups (˜0.3 mmol of sulfate half ester groups, according to the provider).

The DOX removal capacity of ENCC or CNC, qe, defined as the amount of removed drug per unit mass of the nanoadsorbent is presented versus the drug equilibrium concentration in FIG. 2b. By increasing the DOX concentration, initially, most of the drug was captured independent of its concentration, resulting in a low equilibrium drug concentration. As the drug concentration increased, ENCC was saturated, and the equilibrium drug concentration significantly increased. The plateau in FIG. 2b shows the maximum removal capacity of ENCC, i.e., >5200 μg (˜9 μmol) drug per 1 mg of ENCC, which has 5.98 μmol of carboxylate. The DOX removal capacity of ENCC is higher than the stochiometric value, possibly because DOX molecules in solution tend to aggregate.(65) Fitting qe-Ce data to the Langmuir isotherm(66) according to the following equation (Eqn. 1):(67)

$$q\_e = 1 / ( K / C\_e + 1 / q\_m ) \qquad Eqn . 1$$

yields an equilibrium constant K=τads/τads ˜00066 and a maximum removal capacity qm ˜5899 μg mg-1, with the coefficient of determination R2 ˜0.878 Note that τads and τdes are the adsorption and desorption characteristic times, respectively. The drug removal capacity of CNC, shown in FIG. 2b remains below ˜1000 μg mg-1, which is significantly lower than ENCC.

Note that the increase in the removal capacity of CNC at high DOX concentration is possibly due to the aggregation of DOX,(65) and not the CNC effect, as reflected in the near-zero removal capacity at DOX concentrations below 500 mg mL−1. Such a striking difference attests to the superiority that the anionic hairs impart to ENCC, enabling it to perform as a potential nanosponge for biodetoxification applications.

FIG. 2c shows the images of DOX solutions after adding ENCC (right) compared with the ENCC-free DOX solutions (left). Regardless of the DOX/ENCC ratio within the molar ratio of DOX:COO— of ENCC˜0.47-2.07, ENCC effectively precipitates the drug, decreasing the drug concentration in the supernatant. When the ratio of DOX concentration to the COO— of ENCC is less than 1 mol mol-1, the supernatant is almost clear, turning red at higher drug concentrations due to the free, uncaptured drug. The kinetics of drug capture was studied by measuring the DOX concentration in the supernatant over time. FIG. 2d shows the DOX removal percentage versus time. In less than 3 min, the drug removal equilibrates, which shows an extremely fast (almost instantaneous) drug capture. In fact, the first few minutes post nanoadsorbent-drug contact were associated with manually processing the sample and measuring the drug concentration. In the absence of such operator-related time limit, the interaction between ENCC and DOX is immediate (within seconds) as visually seen in FIG. 2c.

The adsorption of DOX to ENCC was examined by studying the colloidal properties of drug aggregates upon ENCC addition. FIG. 2e shows the hydrodynamic size and ζ-potential of DOX aggregates. Before ENCC addition, the drug aggregates have a hydrodynamic size of ˜66 nm and ζ-potential ˜+24 mV, which are consistent with literature.(65) When ENCC was added, the size significantly increased to >350 nm due to the DOX-mediated ENCC aggregation. The ratio of DOX to the COO— of ENCC was maintained low (˜0.1) in a way that ENCC remained excess, i.e., it was not saturated, and no precipitation was formed. The C-potential of ENCC-DOX complexes dropped to ˜−32 mV, which was due to the negatively charged, stable DOX-ENCC aggregates.

### Effects of pH and Ionic Strength on Chemotherapy Drug Capture

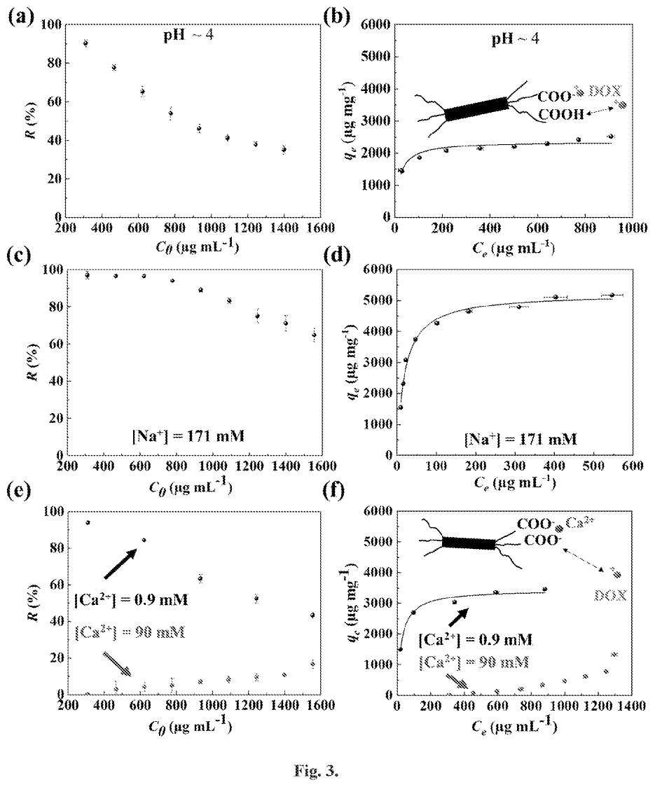

To shed light on the mechanism of ENCC-mediated drug capture, the effects of pH and ionic strength were investigated. FIG. 3 presents the drug removal capability of ENCC at a low pH as well as in the presence of mono- or divalent cations. FIG. 3a shows the drug removal percentage at pH ˜4. The removal immediately starts to decrease as the concentration of drug increases. At this pH, some of the carboxylate groups of ENCC are protonated and therefore cannot interact with the positively charged drug. The adjacent carboxylates of ENCC located on the C2 and C3 of opened glucose ring have pKa ˜4.8-5.6 and 8, which is also reported in our previous work.(67) According to Eqn. 2,

$$pH = pKa + log ( ( ( COO - ) / ( COOH ) ) = 4 \qquad ( 2 )$$

almost all of the COOH groups with pKa=8 are protonated, and only ˜25% of the carboxylate groups with pKa ˜4.8 are deprotonated. Therefore, at pH 4, only 12.5% of the acidic groups (˜0.75 mmol COO— per gram of ENCC) are deprotonated. FIG. 3b presents the removal capacity of ENCC at pH˜4. At this pH, the removal capacity of ENCC is reduced to <2400 μg mg-1, less than half of the removal capacity at pH=7.4, which is higher than the stoichiometric ratio. The Langmuir fit yields K˜0.0080 and qm ˜2365 μg mg-1, with R2˜0.907. This K is higher than the K in DPBS, indicating that the adsorption occurs slower (larger τads/τdes). Besides the aggregation of DOX, the excess DOX may partially shift the equilibrium of carboxylic acid according to the Le Chatelier's principle, increasing the removal capacity beyond the stoichiometric ratio. Such an effect has previously been reported for the interaction between copper ions with ENCC.(52)

The concentration of sodium (Na+) in blood is ˜135-145 mM (31,68) The effect of monovalent ions, such as Na+ on the drug removal was investigated in FIGS. 3c and 3d. At a Na+ concentration above the physiological level (171 mM), the DOX removal percentage (FIG. 3c) and capture capacity (FIG. 3d) were similar to the Na+-free medium (FIGS. 2a and 2b). The presence of monovalent ions in the media does not block the ENCC charge and only shrinks the electrical double layer around the protruding dicarboxylate-modified cellulose chains. Therefore, sodium and likely other monovalent ions in the blood do not hinder the drug capture capacity of ENCC. The Langmuir fit yields K˜0.0036 and qm 5225 μg mg−1, with R2-0.986. FIGS. 3e and 3f present the drug removal percentage and capacity of ENCC in the presence of 0.9 or 90 mM of a divalent cation, in this case calcium (Ca2+). The removal percentage immediately decreased when the divalent ion was added to the medium due to the calcium-mediated charge neutralization of ENCC.(69) At 0.9 mM of Ca2+, the Langmuir fit yielded K˜0.0075 and qm˜3440 μg mg-1, with R2˜0.981, and when the calcium ions reached 90 mM, the removal capacity was <1000 μg mg-1. In contrast to Na+, Ca2+ competes with the positively charged drug molecules in binding to the COO— of ENCC, decreasing the drug capture capacity of ENCC.

### Effect of Plasma Proteins on Chemotherapy Drug Capture

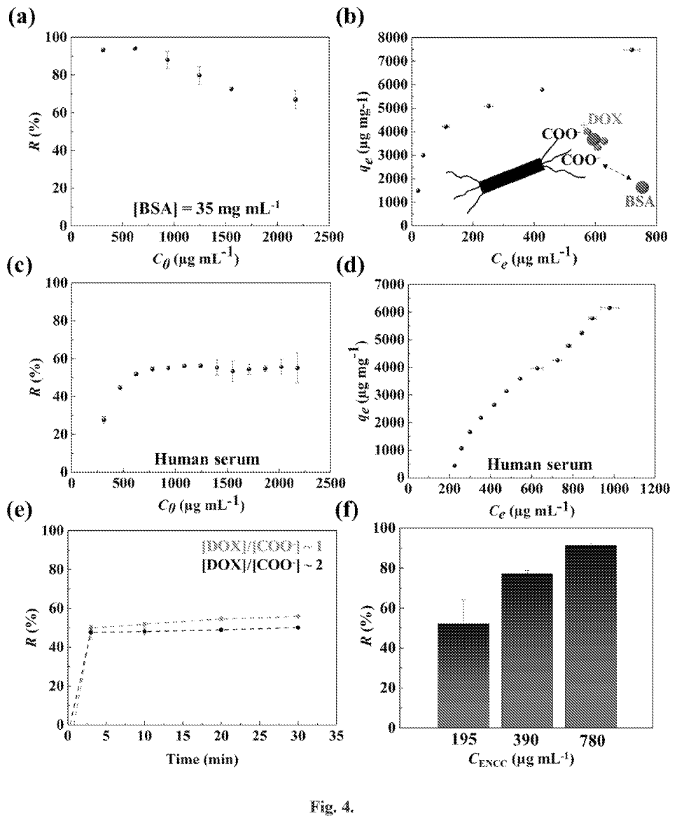

An important constituent of blood is plasma proteins. We evaluated the drug capture capacity of ENCC in the presence of physiologically relevant concentrations of BSA (35 mg mL−1)(70) as a model system for serum albumin. The drug removal percentage and capacity in the presence of BSA are shown in FIGS. 4a and 4b, respectively. The removal percentage remained >90° % up to a drug concentration <1000 μg mL−1, which is higher than the removal percentage in the absence of BSA (FIG. 1a). Compared to the BSA-free system (FIG. 1b), the removal capacity (FIG. 4b) was also enhanced in the presence of BSA and reached >7500 μg mg-1 of ENCC The underlying mechanism for BSA-mediated enhanced drug removal can be explained via the attractive interactions between the positively charged drug and negatively charged BSA:(71) the drug is adsorbed by BSA, which is then picked up by ENCC Accordingly, ENCC picks up the drug-BSA complex, which may include more drugs than the stoichiometric capacity of ENCC in a BSA-free system. This may help overcome the Ca2+-mediated reduction of DOX capture capacity of ENCC.

We investigated the drug capture capacity of ENCC in a physiologically relevant medium, i.e., human serum. FIGS. 4c and 4d show the drug removal percentage and capacity of ENCC in human serum, respectively. The drug removal percentage remained at 60% when the initial drug concentration was increased beyond 500 μg mL⁻¹. As more drug was added to the media, more drug was removed, possibly as a result of drug-protein complex formation, followed by the ENCC-mediated capture. Interestingly, at low drug concentrations (<500 μg mL⁻¹), the removal percentage was noticeably low, which may be due to the stable, negatively charged drug-protein complexes, remaining in the solution. The drug removal capacity of ENCC in human serum shown in FIG. 4d increased by increasing the drug concentration, which may be attributed to the effect of serum proteins. Increasing the drug concentration, facilitates the drug-protein complex formation, decorating the proteins with more positively charged DOX molecules, which are then adsorbed to ENCC. Human serum is typically comprised of proteins (e.g albumin, globulins, and fibrinogen), hormones and enzymes, lipids and carbohydrates.(68,72) Accordingly, while the divalent ions may have negative effects on the drug capture capacity of ENCC, the presence of a range of proteins in the serum enhance the drug removal.

The kinetics of drug capture in human serum is shown in FIG. 4e, suggesting that the capture took place almost immediately, similar to the non-physiological medium (FIG. 2d). To increase the removal percentage of drug, the amount of ENCC was increased. FIG. 4f shows the effect of ENCC content on the drug removal percentage in human serum. The drug removal percentage can readily be increased from ˜50% to >90% by increasing the ENCC concentration from 195 to 780 μg mL⁻¹. Accordingly, ENCC can successfully remove the majority of chemotherapy drug from the serum

### Cytotoxicity Assessment of Hairy Cellulose Nanocrystals

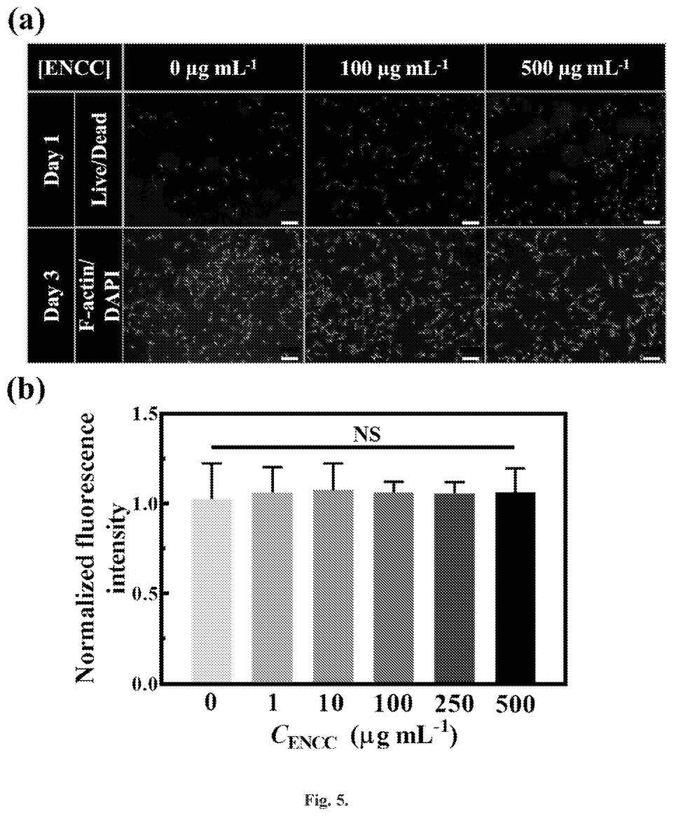

The cytotoxicity of ENCC against human umbilical vein endothelial cells (HUVECs) was examined in vitro. FIG. 5 presents the live/dead and F-actin/DAPI staining and metabolic activity of HUVECs post exposure to varying concentrations of ENCC compared with the ENCC-free control samples. As can be seen in FIG. 5a, the incubation of up to 500 μg of ENCC per mL of cell culture medium yielded similar cell viability as the ENCC-free system after 24 h. none of which resulted in significant cell death. Staining the cytoskeleton and nuclei of cells with F-actin and DAPI, respectively, showed that the ENCC did not have any adverse effects on the cell cytoskeleton and nuclei, e.g., cells spread successfully, and the nuclei remained intact. FIG. 5b shows that after 3 days of exposure to up to 500 μg of ENCC per mL of cell culture medium, the normalized fluorescent intensity, a representation of cell metabolic activity according to the PrestoBlue™ cell viability reagent, remained comparable to that of ENCC-free cells, which underwent no significant loss. Accordingly, ENCC does not impose any toxicity risk to HUVECs, enabling it to effectively interact with the drug.

### Hemolytic Effects of Hairy Cellulose Nanocrystals

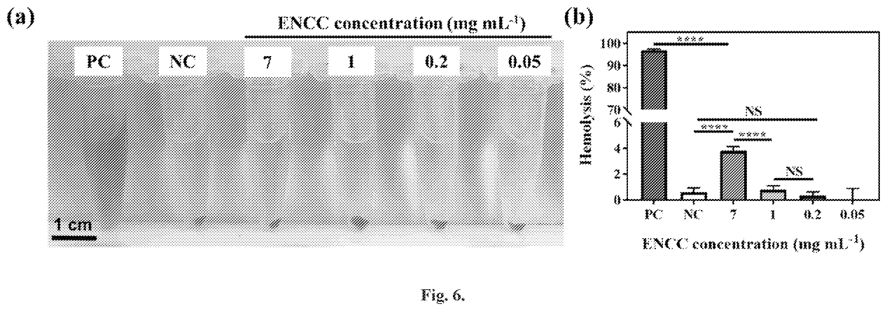

The interaction between ENCC and red blood cells (RBCs) was studied via a standard hemolysis assay (see Materials and Methods). FIG. 6a shows the optical images of blood exposed to ENCC, which was compared to the negative control (NC, PEG) and positive control (PC, Triton X-100). PEG does not induce significant hemolysis, whereas Triton X-100 results in severe RBC damage and hemoglobin leakage, which can be seen in the stable red color of supernatant post-centrifugation. After centrifuging the blood exposed to up to 7 mg mL−1 of ENCC, the supernatant looks as clear as the negative control, showing no noticeable RBC damage. FIG. 6b presents the hemolysis percentage for varying ENCC concentrations, which are compared with the NC and PC. The PC almost fully destroys the RBCs, releasing the hemoglobin, resulting in >95% hemolysis. The ENCC-mediated hemolysis remained less than 4% up to 7 mg mL−1 of ENCC, which is below the maximum percentage allowed, i.e., 5%. This concentration translates into capturing >42 mg of DOX by using only 1 mL of a 0.7% ENCC dispersion without any adverse effects on the cell viability and RBC integrity.

### Hairy Cellulose Nanocrystal-Enabled Biodetoxification

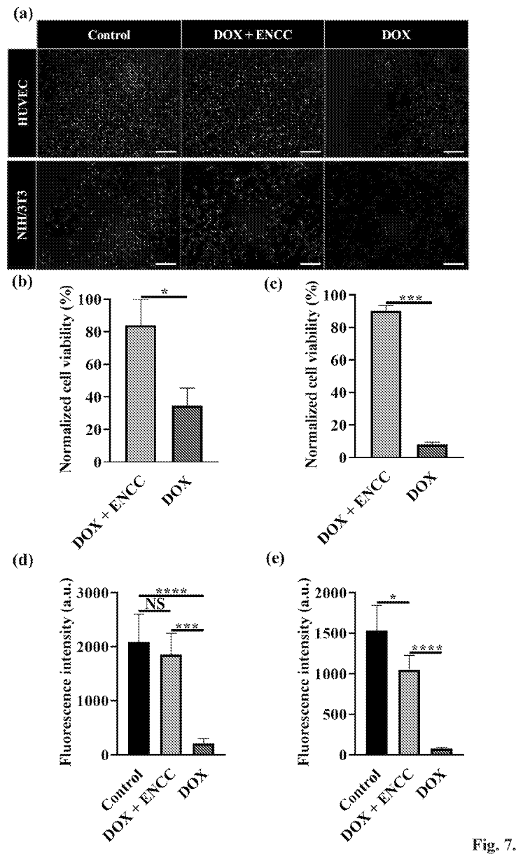

The biodetoxification effect of ENCC was evaluated in vitro using two cell lines. HUVECs and NIH/3T3 cells were cultured to attach to the well plates (2D), followed by adding DOX-containing media pre- and post ENCC treatment and culturing for 8 h. FIG. 7 presents the Live/Dead staining, quantification of cell viability, and the metabolic activity of cells post exposure to DOX with and without ENCC-mediated detoxification. When the cells were exposed to 400 μg of DOX per mL of cell culture media, their viability was compromised due to the DOX-mediated DNA damage.(73) This is reflected in the red staining of HUVECs and NIH/3T3 fibroblasts in FIG. 7a. When the drug containing media was treated with ENCC, followed by removing the precipitated drug via centrifugation, the majority of cells maintained their viability and the Live staining (green color) showed a similar result as the non-toxified (DOX-free) control. The image analysis enabled us to quantify the cell viability via counting the live and total cell numbers. FIGS. 7b and 7c present the cell viability percentage of HUVECs and NIH/3T3 fibroblasts normalized with the viability of control samples, respectively. Adding the drug to the media decreases the cell viability to ˜35% and <10% for HUVECs and NIH/3T3 cells, respectively, whereas the ENCC-mediated detoxification of media maintains more than 80% of both cells viable. The difference between HUVEC and NIH/3T3 fibroblast viability after DOX treatment can be explained by the higher resistance of HUVECs to DOX compared to 3T3 fibroblast cells. HUVECs can develop a resistance to DOX due to increased P-glycoprotein expression.(74) Furthermore, DOX concentrations above 2.5×10-5 M inhibit fibroblast proliferation and DOX concentrations above 5×10⁻⁵ M induce fibroblast cellular toxicity.(75) As we used DOX solutions with a concentration of 400 μg mL−1 (7×10-4 M), the dose caused high cell toxicity in NIH/3T3 cells, resulting in the lower cell viability compared to HUVECs, which were protected due to their increased resistance. The metabolic activities of HUVECs and NIH/3T3 cells, presented in FIGS. 7d and 7e, respectively, showed that the drug decreased the metabolic activity of these cells by more than 2000%, whereas treating the media with ENCC resulted in no significant decrease in the metabolic activity of HUVECs or only 30% decrease in the metabolic activity of fibroblasts. Together, these results show that the ENCC can protect the cells from drug-associated damages.

### CONCLUSIONS

Preventing the systemic circulation of localized drugs, e.g., in localized cancer chemotherapy, can help reduce the drug side effects while allowing frequent, high-dosage treatments. Blood as the main carrier of drugs is particularly challenging to treat due to the existence of a broad range of biomolecules and cells. Developing materials that can specifically capture clinically-relevant concentrations of chemotherapy drugs at physiological conditions without imposing adverse effects on cells is of particular challenge. We have nanoengineered cellulose fibers to produce biocompatible anionic hairy cellulose nanocrystals, known as electrosterically stabilized nanocrystalline cellulose (ENCC), that remove more than 6000 μg of DOX, one of the most common chemotherapy drugs, per one mg of nanoparticles. This is more than 3200% improvement compared to some of the most recent nanoadsorbents, such as DNA-based materials. The ENCC immediately separates the drug from the physiological media, enabling the fast, high-efficiency, and biocompatible biodetoxification of blood in vitro/ex vivo). This technology can set the stage for next generation low-cost drug capture devices.

### REFERENCES

(1) Cancer Statistics, Natl. Cancer Inst. (2020). https-t/www.cancer.gov/about-cancer/understanding/statistics.
(2) A. Pearce, M. Haas, R. Viney, S.-A. Pearson, P. Haywood, C. Brown, R. Ward, Incidence and severity of self-reported chemotherapy side effects in routine care. A prospective cohort study, PLoS One. 12 (2017) e0184360 doi:10.1371/journal.pone.0184360.
(3) R. L. Siegel, K. D. Miller, H. E. Fuchs, A. Jemal, Cancer Statistics, 2021, CA. Cancer J. Clin. 71 (2021) 7-33. doi:https://doi.org/10.3322/caac.21654.
(4) A. D. Waldman, J. M. Fritz, M. J. Lenardo, A guide to cancer immunotherapy: from T cell basic science to clinical practice, Nat. Rev. Immunol 20 (2020) 651-668. doi:10.1038/s4l577-020-0306-5.
(5) K. Shao, S. Singha, X. Clemente-casares, S. Tsai, Y. Yang, Nanoparticle-based immunotherapy for cancer, ACS Nano 9 (2015) 16-30. doi:10.1021/nn5062029
(6) J. Shen, Z. Lu, J. Wang, T. Zhang, J. Yang, Y. Li, G. Liu, X. Zhang, Advances of Nanoparticles for Leukemia Treatment, ACS Biomater. Sci. Eng. 6 (2020) 6478-6489.doi:10.1021/acsbiomaterials.0c01040.
(7) C. Rodriguez-Nogales, Y. González-Fernández, A. Aldaz, P. Couvreur, M. J. Blanco-Prieto, Nanomedicines for pediatric cancers, ACS Nano 12 (2018) 7482-7496. doi:10.1021/acsnano.8b03684.
(8) A. Ediriwickrema, W. M. Saltzman, Nanotherapy for Cancer: Targeting and Multifunctionality in the Future of Cancer Therapies, ACS Biomater. Sci. Eng. 1 (2015) 64-78. doi:10.1021/ab500084 g.
(9) FDA, Daptomycin Highlights of Prescribing Information, 2020
(10) FDA, Cisplatin Package Insert, 2009.
(11) FDA, Fluorouracil Highlights of Prescribing Information, 2016.
(12) R. De Souza, P. Zahedi, C. J. Allen, M Piquette-Miller, Polymeric drug delivery systems for localized cancer chemotherapy, Drug Deliv 17 (2010) 365-375 doi 10.319/107S17541003762854.
(13) J. M. Llovet, J. Bruix, Systematic review of randomized trials for unresectable hepatocellular carcinoma. chemoembolization improves survival, Hepatology. 37 (2003) 429-442. doi:10.1053/jhep.2003.50047.
(14) T. J. Vogl, N. N. N. Naguib, N.-E. A. Nour-Eldin, P Rao, A. H. Emami, S. Zangos, M. Nabil, A. Abdelkader, Review on transarterial chemoembolization in hepatocellular carcinoma: palliative, combined, neoadjuvant, bridging, and symptomatic indications, Eur. J. Radiol. 72 (2009) 505-516. doi:10.1016/j.ejrad.2008.08.007.
(15) N. Imai, M Ishigami, Y. Ishizu, T. Kuzuya, T. Honda, K. Hayashi, Y Hirooka, H. Goto, Transarterial chemoembolization for hepatocellular carcinoma: A review of techniques, World J. Hepatol. 6 (2014) 844. doi:10.4254/wjh.v6.i12.844.
(16) A. Massmann, T. Rodt, S. Marquardt. R. Seidel, K. Thomas, F. Wacker, G. M. Richter, H. U. Kauczor, A. Bicker, P. L. Pereira, Transarterial chemoembolization (TACE) for colorectal liver metastases—current status and critical review, Langenbeck's Arch. Surg. 400 (2015) 641-659. doi:10.1007/s00423-015-1308-9.
(17) R. Sacco, G. Tapete, N. Sim onetti, R. Sellitri, V. Natali, S. Melissari, G. Cabibbo, L. Biscaglia, G. Bresci, L. Giacomelli, Transarterial chemoembolization for the treatment ofhepatocellularcarcinoma: a review, J. Hepatocell. Carcinoma. 4(2017) 105. doi:10.2147/JHC.S103661.
(18) K. Y. Tam, K. C. F. Leung, Y. X. J. Wang, Chemoembolization agents for cancer treatment, Eur. J. Pharm. Sci. 44 (2011) 1-10. doi.10.1016/j.ejps.2011.06.013.
(19) Y. Ichikawa, M. Ghanefar, M. Bayeva, R. Wu, A. Khechaduri, S. V. N. Prasad, R. K. Mutharasan, T. J. Naik, H. Ardehali, Cardiotoxicity of doxorubicin is mediated through mitochondrial iron accumulation, J. Clin Invest 124 (2014) 617-630, doi:10.1172/JC172931.
(20) D. Jain, Cardiotoxicity of doxorubicin and other anthracycline derivatives, J. Nucl. Cardiol. 7 (2000) 53-62. doi:10.10671mnc.2000.103324
(21) C Yee. D. McCoy, J. Yu, A. Losey, C. Jordan, T. Moore, C. Stillson, H. J. Oh, B. Kilbride, S. Roy, A. Patel, M. W. Wilson, S. W. Hetts, Endovascular Ion Exchange Chemofiltration Device Reduces Off-Target Doxorubicin Exposure in a Hepatic Intra-arterial Chemotherapy Model, Radiol. Imaging Cancer. 1 (2019)el90009. doi:10.1148/rycan 2019190009.
(22) C M. Blumenfeld, M. D. Schulz, M. S. Aboian, M W. Wilson, T. Moore, S. W. Hetts, R. H. Grubbs, Drug capture materials based on genomic DNA-functionalized magnetic nanoparticles, Nat. Commun. 9 (2018) 1-7. doi:10.1038/s41467-018-05305-2
(23) D. W. Yee, S. W. Hetts, J. R. Greer, 3D-Printed Drug Capture Materials Based on Genomic DNA Coatings, ACS Appl. Mater Interfaces. (2021) doi:10.1021/acsami.lc05209.
(24) X. C. Chen, H. J. Oh, J. F. Yu, J. K. Yang, N. Petzetakis, A. S. Patel, S. W.

Hetts, N. P. Balsara, Block Copolymer Membranes for Efficient Capture of a Chemotherapy Drug, ACS Macro Lett. 5 (2016) 936-941. doi:10.102l/acsmacrolett.6b00459.
(25) H. J. Oh, M. S. Aboian, M. Y. J. Yi, J. A. Maslyn, W. S. Loo, X. Jiang, D. Y. Parkinson, M. W. Wilson, 1. Moore, C. R. Yee, G. R. Robbins, F. M. Barth, J. M. Desimone, S. W. Hetts, N. P. Balsara, 3D Printed Absorber for Capturing Chemotherapy Drugs before They Spread through the Body, ACS Cent. Sci. 5 (2019) 419-427 doi:10.1021/acscentsci.8b00700.
(26) T. M. Karpiński, Selected medicines used in iontophoresis, Pharmaceutics. 10 (2018). doi-10.3390/pharmaceutics10040204.
(27) K. Rausch, A. Reuter, K. Fischer, M. Schmidt, Evaluation of nanoparticle aggregation in human blood serum, Biomacromolecules. 11 (2010) 2836-2839. doi:10.1021/bm100971q.
(28) S. Behzadi, V. Serpooshan, W. Tao, M. A. Hamaly, M. Y. Alkawareek, E. C. Dreaden, D. Brown, A. M. Alkilany, O. C. Farokhzad, M. Mahmoudi, Cellular uptake of nanoparticles: journey inside the cell, Chem. Soc. Rev. 46 (2017) 4218-4244, doi:10.1039/c6cs00636a.
(29) M. R. Buchakjian, T. Ginader, K. K. Tasche, N. A. Pagedar, B. J. Smith, S. M. Sperry, PEGylation as a strategy for improving nanoparticle-based drug and gene delivery, Adv. Drug Deliv. Rev. 99 (2018) 28-51. doi:10.1177/0194599818773070.
(30) Y. Liu, M. K. Shipton, J. Ryan, E. D. Kaufman, S. Franzen, D. L Feldheim, Synthesis, stability, and cellular internalization of gold nanoparticles containing mixed peptide-poly(ethylene glycol) monolayers, Anal. Chem. 79 (2007) 2221-2229 doi:10.1021/ac061578f
(31) T. L. Moore, L. Rodriguez-Lorenzo, V. Hirsch, S. Balog, D. Urban, C. Jud, B. Rothen-Rutishauser, M. Lattuada, A. Petri-Fink, Nanoparticle colloidal stability in cell culture media and impact on cellular interactions, Chem. Soc. Rev. 44 (2015) 6287-6305. doi:10.1039/c4cs00487f.
(32) T. L. Doane, C.-H. Chuang, R. J. Hill, C. Burda, Nanoparticle ζ-potentials, Acc. Chem. Res. 45 (2012) 317-326. doi:10.1021/ar200 (13c.
(33) J.-H. Kim, B. S. Shim, H S. Kim, Y.-J. Lee, S.-K. Min, D. Jang, Z. Abas, J. Kim, Review of nanocellulose for sustainable future materials, Int. J. Precis. Eng. Manuf. Technol 2 (2015) 197-213. doi:10.1007/s40684-015-0024-9.
(34) C. Endes, S. Camarero-Espinosa, S. Mueller, E. J. Foster, A. Petri-Fink, B. Rothen-Rutishauser, C. Weder, M. J. D. Clift, A critical review of the current knowledge regarding the biological impact ofnanocellulose, J. Nanobiotechnology. 14 (2016) 78. doi:10.1186/s12951-016-0230-9.
(35) M. Roman, Toxicity of Cellulose Nanocrystals: A Review, Ind. Biotechnol. 11 (2015) 25-33. doi:10.1089/ind.2014.0024.
(36) M. J. D. Clift, E. J. Foster. D. Vanhecke, D. Studer, P. Wick, P. Gehr, B Rothen-Rutishauser, C. Weder, investigating the interaction of cellulose nanotibers derived from cotton with a sophisticated 3D human lung cell coculture, Biomacromolecules. 12 (2011) 3666-3673. doi:10.1021/bm200865j.
(37) N. Yanamala, M. K. Farcas, M K Hatfield, E. R. Kisin, V. E. Kagan, C. L Geraci, A. A. Shvedova, In vivo evaluation of the pulmonary toxicity of cellulose nanocrystals: A renewable and sustainable nanomaterial of the future, ACS Sustain. Chem. Eng. 2(2014) 1691-1698 doi:10.1021/sc50053k.
(38) R. M A Domingues, M. E. Gomes, R. L Reis, The potential of cellulose nanocrystals in tissue engineering strategies, Biomacromolecules. 15 (2014) 2327-2346. doi:10.1021/bm500524s.
(39) R. T. Cullen, B. G. Miller, A. D. Jones, J. M. G. Davis, Toxicity of cellulose fibres, Ann. Occup. Hyg. 46 (2002) 81-84. doi: 10.1093/annhyg/46.suppl-1.81.
(40) A. Sheikhi, J. Hayashi, J. Eichenbaum, M. Gutin, N. Kuntjoro, Recent advances in nanoengineering cellulose for cargo delivery, J. Control. Release. 294 (2019) 53-76. doi:10.1016/j.jconrel 2018.11.024.
(41) J. H. Cummings, Cellulose and the human gut., Gut. 25 (1984) 805-810. doi:10.1136/gut.25.8.805.
(42) R. J Moon, A. Martini, J. Nairn, 3. Simonsen, J. Youngblood, Cellulose nanomaterials review: structure, properties and nanocomposites, Chem. Soc. Rev. 40 (2011) 3941-3994 doi:10.1039/ccsOi008b.
(43) D. Roy, M. Semsarilar, J. T. Gutthrie, S. Perrier, Cellulose modification by polymer grafting a review, Chem. Soc. Rev 38 (2009) 2046-2064. doi:10.1039/B808639G.
(44) E. J. Foster, R. J. Moon, U. P. Agarwal, M. J. Bortner, J. Bras, S. Carnarero-Espinosa, K. J. Chan, M. I.D. Clift, E. D. Cranston, S J Eichhom, Current characterization methods for cellulose nanomaterials, Chem. Soc. Rev. (2018). doi:10.1039/c6cs00895j.
(45) H. Tao, N. Lavoine, F. Jiang, J. Tang, N. Lin, Reducing end modification on cellulose nanocrystals: Strategy, characterization, applications and challenges, Nanoscale Horizons. 5 (2020) 607-627. doi:10.1039%/d0nh00016 g.
(46) H. Zhu, W. Luo, P N. Ciesielski, Z. Fang, J. Y. Zhu, G Henriksson, M. E. Himmel, L Hu. Wood-Derived Materials for Green Electronics, Biological Devices, and Energy Applications, Chem. Rev. 116 (2016) 9305-9374. doi:10.1021/acs. chemrev.6b00225.
(47) D. Klemm, F. Kramer, S Moritz, T. Lindstrom, M. Ankerfors, D. Gray, A. Dorris, Nanocelluloses: A new family of nature-based materials, Angew. Chem. Int. Ed. 50 (2011) 5438-5466. doi:10.1002/anie.201001273.
(48) A. Sheikhi, Emerging cellulose-based nanomaterials and nanocomposites, in: Nanomater. Polym. Nanocomposites, Elsevier, 2019: pp. 307-351. doi:10.1016/B978-0-12-814615-6.00009-6.
(49) T. G. M. van de Ven, A. Sheikhi, Hairy cellulose nanocrystalloids: A novel class of nanocellulose, Nanoscale. 8 (2016) 15101-15114. doi:10.1039/c6nr01570k.
(50) A. Sheikhi, H. Yang, M. N. Alam, T. G. M. van de Ven, Highly stable, functional hairy nanoparticles and biopolymers from wood fibers. Towards sustainable nanotechnology, J. Vis. Exp. 113 (2016) e54133. doi:10.3791/54133.
(51) A. Sheikhi, T. G. M. van de Ven, Colloidal aspects of Janus-like hairy cellulose nanocrystalloids, Curr. Opin. Colloid Interface Sci. 29 (2017) 21 . . . 31. doi:http://dx.doi.org/10.1016/j cocis.2017.02.001.
(52) A. Sheikhi, S. Safari, H. Yang, T. G. M. van de Ven, Copper removal using electrosterically stabilized nanocrystalline cellulose, ACS Appl. Mater. Interfaces. 7 (2015) 11301-11308 doi:10.1021/acsami.5b01619.
(53) S. Y. van der Zanden, X. Qiao, J. Neefjes, New insights into the activities and toxicities of the old anticancer drug doxorubicin, FEBS J. (2020) doi:10.1111/febs.15583.
(54) A. Pugazhendhi, T. N. J.1. Edison, B. K. Velmurugan, J. A. Jacob, 1. Karuppusamy, Toxicity of Doxorubicin (Dox) to different experimental organ systems, Life Sci 200 (2018) 26-30 doi:10.1016/j. lfs.2018.03.023.
(55) H. G. Keizer, H. M Pinedo, G. J. Schuurhuis, 1.1 Joenje, Dcrorubicin (adriamycin): a critical review of free radical-dependent mechanisms of cytotoxicity, Pharmacol. Ther. 47 (1990) 219-231. doi:10.1016/0163-7258(90)90088-J
(56) M. Songbo, H. Lang, C. Xinyong, X. Bin, Z. Ping, S. Liang, Oxidative stress injury in doxorubicin-induced cardiotoxicity, Toxicol Lett. 307 (2019) 41-48. doi:10.1016/j.toxlet.2019.02.013.
(57) H. Yang, A. Tejado, N. Alam. M. Antal, T. G. M. van de Ven, Films prepared from electrosterically stabilized nanocrystalline cellulose, Langmuir. 28 (2012) 7834-7842. doi:10.1021/la2049663.
(58) J. Araki, M. Wada, S. Kuga, Steric stabilization of a cellulose microcrystal suspension by poly (ethylene glycol) grafting, Cellulose. 17 (2001)21-27. doi:10.1021/1a001070m.
(59) M. Tavakolian, H. Wiebe, M. A. Sadeghi, TGM. Van De Ven, Dye Removal Using Hairy Nanocellulose: Experimental and Theoretical Investigations, ACS Appl. Mater. Interfaces. 12 (2020) 5040-5049. doi:10.102l/acsami.9b18679.
(60) D. Netas, P. Klapetek, Gwyddion: an open-source software for SPM data analysis, Open Phys. 10 (2012) 181-188. doi:10.2478/s11534-011-0096-2.
(61) ASTM E2524—08 Standard Test Method for Analysis of lemolytic Properties of Nanoparticles, ASTM Int. (2008).
(62) C. A. Schneider, W. S. Rasband, K. W. Eliceiri, NIH Image to ImageJ: 25 years of image analysis, Nat. Methods. 9 (2012) 671-675. doi.10.1038/nmeth.2089
(63) C. Fraschini, G. Chauve, J. Bouchard, TEMPO-mediated surface oxidation of cellulose nanocrystals (CNC's), Cellulose. 24 (2017) 2775-2790 doi:10.1007/s10570-0l7-1319-5.
(64) P. Lu, Y. Lo Hsieh, Preparation and properties of cellulose nanocrystals: Rods, spheres, and network, Carbohydr. Polym. 82 (2010) 329-336. doi:10.1016/j.carbpol 0.2010.04.073
(65) Z. Folop, R. Gref, T Lofisson, A permeation method for detection of self-aggregation of doxorubicin in aqueous environment, Int. J. Pharm. 454(2013)559-561 doi:10.1016/j.ijpharn.2013.06.058.
(66) 1. Langmuir, The adsorption of gases on plane surfaces of glass, mica and platinum., J Am. Chem. Soc. 40 (1918) 1361-1403.
(67) H. Yang, A. Sheikhi, T. G. M. van de Ven, Reusable Green Aerogels from Cross-Linked Hairy Nanocrystalline Cellulose and Modified Chitosan for Dye Removal, Langmuir. 32 (2016) 11771-11779. doi:10.1021/acs.langmuir.6b03084.
(68) L. M. Biga, S. Dawson, A. Harwell, R. Hopkins, J. Kaufmann, M. LeMaster, P. Matern, K. Morrison-Graham, D. Quick, J. Runyeon, Anatomy & Physiology, in: Anat. Physiol, 2017: pp. 1054-1063, 1727-1737 doi:10.5399/osu/1116.
(69) A. Sheikhi, A. Kakkar, T. G. M. van de Ven, A leaf out of Nature's book: hairy nanocelluloses for bioinspired mineralization, Cryst. Growth Des 16 (2016) 4627-4634. doi.10.1021/acs.cgd.6b00713.
(70) T. Peters, All about albumin: biochemistry, Genet. Med. Appl. (Academic Press. 1996). (1996).
(71) D. Fologea, B. Ledden, D. S. McNabb, J. Li, Electrical characterization of protein molecules by a solid-state nanopore, Appl. Phys. Lett. 91 (2007) 1-9. doi:10.1063/1.2767206.
(72) N. Psychogios, D. D. Hau, J. Peng, A. C. Guo, R. Mandal, S. Bouatra, I. Sinelnikov, R. Krishnamurthy, R. Eisner, B. Gautam, N. Young, J. Xia, C. Knox, E. Dong, P. Huang, Z. Hollander, T. L. Pedersen, S R. Smith, F. Bamforth, R. Griner, B. McManus, J. W. Newman, T. Goodfriend, D. S. Wishart, The human serum metabolome, PLoS One. 6 (2011). doi:10.1371/jounal.pone.0016957.
(73) H.-S. Kim, Y.-S. Lee, D.-K. Kim, Doxorubicin exerts cytotoxic effects through cell cycle arrest and Fas-mediated cell death, Pharmacology. 84 (2009) 300-309. doi:10.1159/000245937.
(74) L. Huang, C. Perrault, J. Coelho-Martins, C. Hu, C. Dulong, M. Varna, J. Liu, J. Jin, C. Soda, L. Cazin, Induction of acquired drug resistance in endothelial cells and its involvement in anticancer therapy, J. Hematol. Oncol. 6 (2013) 49. doi:10.1186/1756-8722-6-49
(75) L. Izaguirre, I. Pinilla, F. Gonzalvo, S. Perez, F. M. Honrubia, Effect of doxorubicin on fibroblast migration and proliferation, Ann. Ophthalmol. 35 (2003) 48-52. doi:10.1385/AO:35:1:48.

All publications mentioned herein (e.g. those disclosed above, Young et al., Mater Today Chem. 2022 March; 23:100711. doi: 10.1016/j.mtchem.2021.100711. Epub 2021 Dec. 30; US Patent Publication 20170340797 and Van de Ven et al., Nanoscale, 2016, 8, 15101-15114; Campano C, Lopez-Exposito P, Blanco A, Negro C, van de Ven TGM. Hairy cationic nanocrystalline cellulose as a novel flocculant of clay. J Colloid interface Sci. 2019 Jun. 1; 545:153-1l61. doi: 10.1016/j.jcis.2019.02.097.Epub2019 Mar. 1. PMID: 30878782; Yang, H., van de Ven, T. G. M. Preparation of hairy cationic nanocrystalline cellulose. Cellulose 23, 1791-1801 (2016). https://doi.org/10.1007/s10570-016-0902-5; Koshani et al, Journal of Colloid and Interface Science Volume 607, Part 1, February 2022, Pages 134-144; Campano, C., Lopez-Exposito, P., Blanco, A. et al Hairy cationic nanocrystalline cellulose as retention additive in recycled paper. Cellulose 26, 6275-6289 (2019); Tavakolian et al., Journal of Colloid and Interface Science, Volume 541, 1 Apr. 2019, Pages 444-453; C. M Blumenfeld, M. D. Schulz, M. S. Aboian, M W. Wilson, T. Moore, S. W. Hetts, R. H. Grubbs, Drug capture materials based on genomic DNA-functionalized magnetic nanoparticles, Nat. Commun. 9 (2018) 1-7 doi-10.1038/s41467-018-05305-2; D. W Yee, S. W. Hetts, J. R. Greer, 3D-Printed Drug Capture Materials Based on Genomic DNA Coatings, ACS Appl. Mater. Interfaces. (2021). doi:10.1021/acsami.1c05209; and H. J. Oh, M. S. Aboian, M. Y J Yi, J A. Maslyn, W. S. Loo, X. Jiang, D. Y. Parkinson, M. W. Wilson, T Moore, C. R. Yee, G. R. Robbins, F. M. Barth, J. M. Desimone, S. W Hetts. N. P. Balsara, 3D Printed Absorber for Capturing Chemotherapy Drugs before They Spread through the Body, ACS Cent. Sci. 5 (2019) 419-427. doi:10.1021/acscentsci.8b00700.) are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A composition of matter comprising electrosterically stabilized nanocrystalline cellulose, wherein the electrosterically stabilized nanocrystalline cellulose binds at least 1000 mg of doxorubicin per gram of electrosterically stabilized nanocrystalline cellulose when the electrosterically stabilized nanocrystalline cellulose is contacted with a fluid comprising doxorubicin.

2. The composition of claim 1, further comprising:
a pharmaceutically acceptable excipient;
a polypeptide having a negative charge at physiological pH;
blood; and/or;
a therapeutic agent.

3. The composition of claim 1, where in the polypeptide comprises an albumin and/or the therapeutic agent comprises doxorubicin.

4. The composition of claim 1, wherein the electrosterically stabilized nanocrystalline cellulose is coupled to a matrix.

5. The composition of claim 1, wherein the electrosterically stabilized nanocrystalline cellulose binds at least 2000 mg, 3000 mg, 4000 mg, 5000 mg, or 6000 mg of doxorubicin per gram of electrosterically stabilized nanocrystalline cellulose when the electrosterically stabilized nanocrystalline cellulose is contacted with a fluid comprising the doxorubicin.

6. The composition of claim 1, wherein the electrosterically stabilized nanocrystalline cellulose is disposed with a depot of a medical device.

7. A medical device comprising
a depot;
a composition of claim 1 disposed with in the depot;
a first conduit having a first end adapted to capture fluid and a second end coupled to the depot.

8. The medical device of claim 7, further comprising a second conduit having a first end coupled to the depot and a second end adapted to deliver a fluid out of the depot.

9. The medical device of claim 7, further comprising a housing which envelops the depot, wherein an exterior surface of the housing comprises a biocompatible material adapted to be inserted in vivo.

10. The medical device of claim 7, wherein the medical device comprises a catheter.

11. The medical device of claim 7, wherein the medical device comprises an elongate body having a proximal end and a distal end.

12. The medical device of claim 7, wherein the electrosterically stabilized nanocrystalline cellulose is coupled to a matrix.

13. The medical device of claim 7, where in the electrosterically stabilized nanocrystalline cellulose within the medical device comprises blood and/or doxorubicin.

14. The medical device of claim 7, wherein the electrosterically stabilized nanocrystalline cellulose binds at least 6000 mg of doxorubicin per gram of electrosterically stabilized nanocrystalline cellulose when the electrosterically stabilized nanocrystalline cellulose is contacted with a fluid comprising the doxorubicin.

15. A method of removing doxorubicin from a fluid comprising the doxorubicin, the method comprising contacting a composition of claim 1 with the fluid such that the electrosterically stabilized nanocrystalline cellulose captures the doxorubicin, thereby removing doxorubicin from the fluid.

16. The method of claim 15, wherein the fluid is blood.

17. The method of claim 16, wherein the blood is obtained from a patient selected to be undergoing a therapeutic regimen comprising the administration of doxorubicin.

18. The method of claim 17, wherein the patient is selected to be on diagnosed as having a cancer of the breast, bladder, kidney, ovary, thyroid, stomach, lung, bone, nerve, tissue, joint or soft tissue.

19. The method of claim 17, wherein the patient is selected to be one diagnosed as having a lymphoma or leukemia.

20. The method of claim 15, wherein the fluid is contacted in vivo.
